(19) United States
(12) Patent Application Publication
(10) Pub. No.: US 2012/0215204 A1
(43) Pub. Date: Aug. 23, 2012

(54) METHOD OF CONTROLLING THE PROPAGATION OF MRSA, STAPH AND OTHER INFECTIONS THAT COLONIZE IN THE NOSE

(76) Inventor: Michael F. Moore, Dunmore, PA (US)

(21) Appl. No.: 13/458,557

(22) Filed: Apr. 27, 2012

Related U.S. Application Data
(63) Continuation-in-part of application No. 13/304,483, filed on Nov. 25, 2011.
Application No. 13/304,483 is a continuation-in-part of application No. 12/906,493, filed on Oct. 18, 2010.
(60) Provisional application No. 61/252,597, filed on Oct. 16, 2009.

(51) Int. Cl. A61M 31/00

(52) U.S. Cl. 604/514; 604/285

(57) ABSTRACT

A method for reducing the spread of bacterial infections includes identifying a group of individuals at risk of an infection that colonizes in the nose, determining whether any individuals can be exclude, designating an individual or group of individuals who will receive treatment, providing a strip, mask, and/or clip, the strip, mask, and/or clip having an effective amount of antimicrobial agent capable of being delivered to the colony of bacteria in, on, and around the nose, the strip, mask, and/or clip is applied to the nose of the individual or group so to allow the agent to migrate to eradicate the colony.

### CROSS REFERENCE TO RELATED APPLICATIONS

This application is a continuation-in-part of, and claims priority to U.S. patent application Ser. No. 13/304,483, filed on Nov. 25, 2011 entitled METHOD OF CONTROLLING THE PROPAGATION OF MRSA, STAPH AND OTHER INFECTIONS THAT COLONIZE IN THE NOSE, U.S. patent application Ser. No. 12/906,493, filed on Oct. 18, 2010 entitled METHOD OF CONTROLLING THE PROPAGATION OF MRSA, STAPH AND OTHER INFECTIONS THAT COLONIZE IN THE NOSE and U.S. provisional patent application entitled METHOD OF CONTROLLING THE PROPAGATION OF MRSA, STAPH AND OTHER INFECTIONS THAT COLONIZE IN THE NOSE, No. 61/252,597 filed on Oct. 16, 2009, and incorporates all of the same by reference as if set forth herein in its entirety.

### STATEMENT AS TO FEDERALLY SPONSORED RESEARCH

Not applicable.

### BACKGROUND OF INVENTION

a. Field of Invention

The invention relates generally to methods for controlling the spread of bacterial infections within health care facilities, skilled nursing facilities, institutional care facilities such as prisons, dormitories, military barracks, or anywhere people come in close proximity—increasing the incidence of the spread of infection among a population. Particularly, the invention relates to a method or protocol for reducing the spread of infection in the abovementioned situations by providing an adhesive strip metalized with silver or other effective antimicrobial agent and applying the strip to the nose for delivery to the colony to eradicate the colonization of bacteria. In an embodiment of the present invention, a surgical mask, or a filtration mask, or a similar mask that covers the nose and mouth is provided. Said mask having a portion of the mask that contacts the nose, and said portion containing an antimicrobial agent capable of being delivered to the nose to eradicate the colonization of bacteria within the nose. In an embodiment of the present invention, a clip is provided. The clip has two arms that contact the lining of the nostrils for delivering an antimicrobial agent to the nasal mucosa.

b. Background of Invention

Methicillin-resistant staphylococcus aureus (MRSA) is a bacterium responsible for difficult-to-treat infections in humans. It may also be referred to as multidrug-resistant staphylococcus aureus or oxacillin-resistant staphylococcus aureus (ORSA). MRSA is by definition a strain of Staphylococcus aureus that is resistant to a large group of antibiotics called the beta-lactams, which include the penicillins and the cephalosporins. The invention is primarily targeted at controlling the spread of MRSA, but is also intended at being effective at other bacterium that colonize in the nose.

MRSA is a resistant variation of the common bacterium Staphylococcus aureus. It has evolved an ability to survive treatment with beta-lactam antibiotics, including methicillin, dicloxacillin, nafcillin, and oxacillin. MRSA is especially troublesome in hospital-associated (nosocomial) infections. In hospitals, patients with open wounds, invasive devices, and weakened immune systems are at greater risk for infection than the general public. The infection may spread in a number of ways. A patient may come to a health care facility having the infection. Since the infection colonizes in the nose, a patient may easily spread the infection by touching or wiping the nose and any other article. Hospital staff who do not follow proper sanitary procedures may transfer bacteria from patient to patient. Visitors to patients with MRSA infections or MRSA colonization are advised to follow hospital isolation protocol by using the provided gloves, gowns, and masks if indicated. Visitors who do not follow such protocols are capable of spreading the bacteria to cafeterias, bathrooms, and elevators.

The organism is often sub-categorized as community-acquired MRSA (CA-MRSA) (community acquired is also associated with a toxin PVL toxin) or health care-associated MRSA (HA-MRSA) (It is also known as Hospital acquired MRSA) although this distinction is complex. Some have defined CA-MRSA by characteristics of patients who develop an MRSA infection while other authors have defined CA-MRSA by genetic characteristics of the bacteria themselves. The first reported cases of community-acquired MRSA began to appear in the mid-1990s from Australia, New Zealand, the United States, the United Kingdom, France, Finland, Canada, and Samoa, notable because they involved people who had not been exposed to a health-care setting. The new CA-MRSA strains have rapidly become the most common cause of cultured skin infections among individuals seeking emergency medical care in urban areas of the United States. These strains also commonly cause skin infections in men who have sex with men, athletes, prisoners and soldiers.

There are two major ways people become infected with MRSA. The first is physical contact with someone who is either infected or is a carrier (people who are not infected but are colonized with the bacteria on their body) of MRSA. The second way is for people to physically contact MRSA on any objects such as door handles, floors, sinks, or towels that have been touched by an MRSA-infected person or carrier. It is known that MRSA colonizes in the nose, and thus in health care settings, patients are sometimes screened for MRSA by taking a culture of the nose. If positive, the patient is treated with nose drops having an effective antimicrobial agent, and subsequent cultures are taken to determine of the infection is eradicated. Problematic with this treatment is that the nose drops are not approved for treatment of pediatric patients and resistant to the antimicrobial nose drops is increasing.

If the patient tests positive, surgical procedures are postponed.

b. Description of Related Art

The following patents are representative of the field pertaining to the present invention:

U.S. Pat. No. 7,087,249 describes the use of one or more antimicrobial metals preferably selected from silver, gold, platinum, and palladium but most preferably silver, formed with atomic disorder, and preferably in a nanocrystalline form, for reducing inflammation or infection of the mucosal membrane. Transdermal patches may provide controlled delivery of the antimicrobial metal to the mucosal membrane. For example, an adhesive patch or adhesive matrix patch, can be prepared from a backing material and an adhesive, such as an acrylate adhesive. Powders or solutions of the antimicrobial metal may be formulated into the adhesive casting solution and allowed to mix thoroughly. The solution is cast directly onto the backing material and the casting solvent is evaporated in an oven, leaving an adhesive film. Alternatively, a poly-urethane matrix patch can be employed to deliver the antimicrobial metal to the mucosal membrane. The layers of this patch comprise a backing, a polyurethane drug/enhancer matrix, a membrane, an adhesive, and a release liner. The polyurethane matrix is prepared using a room temperature curing polyurethane prepolymer. Addition of water, alcohol, and drug to the prepolymer results in the formation of a tacky firm elastomer that can be directly cast onto the backing material.

U.S. patent application Ser. No. 11/101,386 discusses method of preventing or treating staphylococcal bacterial infection in an individual is disclosed. A vaccine based on a conjugate the 336 polysaccharide antigen can be used for active protection in individuals who are to be subjected to conditions that place them at immediate risk of developing a bacterial infection, as would be case in the context of a catheterization or a surgical procedure. Alternatively, antibodies raised in response to the antigen can be used to treat or to provide passive protection to individuals. The method can be used in a population of patients at risk for infection by various species of Staphylococcus or various types of Staphylococcus aureus.

U.S. patent application Ser. No. 11/490,512 discloses a vaccine based on a conjugate of PS1 polysaccharide antigen can be used for active protection in individuals who are to be subjected to conditions that place them at immediate risk of developing a bacterial infection, as would be case in the context of a catheterization or a surgical procedure. Alternatively, antibodies raised in response to the antigen can be used to treat or to provide passive protection to individuals. The method can be used in a population of patients at risk for infection by various species of Staphylococcus or various types of Staphylococcus epidermidis.

U.S. patent application Ser. No. 11/432,876 describes a compression stretch bandage formed from at least one layer of a stretchable, textile material forming a body of the bandage, a base material attached to the stretchable, textile material on a first side, and a silver material attached to the base material for reducing risk of infection. The bandage may be a flexible, stretchable, hydrophilic bandage that reduce the risk of infection at a wound by providing a moist environment that will aid in optimum release of silver ions into the wound.

U.S. patent application Ser. No. 10/836,530 describes a method for enhancing the metal ion release rate of a substrate having a coating of a metal thereon. The method includes the steps of forming the metal-coated substrate and then subjecting the metal-coated substrate to a step that removes portions of the metal coating to form at least one notch in the metal coating, thereby increasing the surface area of the metal coating. The increased surface area enhances the metal ion release rate of the substrate. The metal may be silver. A silver-coated substrate may be used in the formation of medical products having increased antimicrobial and/or anti-fungal characteristics.

The prior art Breathe Rite® nose strips consist of a spring member strip with an adhesive backing. The device enlarges the nasal passageways through the affixing of the flexible adhesive strip, low across the bridge of the nose.

Notwithstanding the prior art, the present invention is neither taught nor rendered obvious thereby.

### SUMMARY OF INVENTION

The invention relates to a method or protocol for reducing the spread of bacterial infections such as MRSA among a population.

In a preferred embodiment of the present invention, the method includes identifying a group of individuals at risk of carrying or becoming carriers of a bacterial infection that colonizes in the nose. Identification step may alternately include identifying an activity or location of an individual that puts said individual at risk of carrying the infection or becoming a carrier of said infection.

An other step may involve determining whether any individuals can be excluded from treatment due to reasons relating to the individual's other activities or precautions taken, which may result in the individual having a lower risk of carrying or becoming a carrier relative to the remaining members of the classification.

An other step involves designating an individual or group of individuals who will receive treatment with the strip based on the identification/exclusion steps above. In some embodiments, treatment is prophylactic in nature, wherein the designated individual or group of individuals is/are not confirmed to be carriers of a bacterial infection that colonizes in the nose.

An other step of the invention includes providing a strip, the strip having an effective amount of antimicrobial agent capable of being delivered from the strip to the colony of bacteria in the nose. The mode of transport may occur dermally (over the skin surface), transdermally, by moisture and/or oils on the nose, or otherwise. Preferably, the antimicrobial agent comprises silver. However, any agent capable of eradicating the bacterial colonization of the nose is suitable for use with the strip. The strip is exteriorly applied to the nose of the designated individual or group of individuals so to allow the antimicrobial agent to interact with the colony to suppress the colonization, or altogether eradicate the colony.

In an embodiment of the present invention there is a mask for covering the nose of a person. The mask comprising an air filtering shield constructed and configured to cover the nose and mouth of a person allowing the person to breathe filtered air through the shield. The shield typically includes a retainer connected to the shield and arrangeable about a person's head with the shield retained against the person's face. Other known retainer means may be employed sufficient to retain the shield against the person's face. The mask includes a nose contacting portion that physically contacts a person's nose. Said nose contacting person contains an effective amount of an antimicrobial agent (as discussed herein) capable of migrating from said nose contacting portion to the colony of bacteria in the person's nose. As discussed herein, the antimicrobial agent is effective to minimize or eradicate bacteria that colonize in the nose.

In an embodiment of the present invention, the nose contacting portion of the shield includes an adhesive strip applied to the shield. The adhesive strip containing an effective amount of an antimicrobial agent (as discussed in greater detail herein) capable of migrating from said nose contacting portion to the colony of bacteria.

In an embodiment of the present invention, the effective amount of an antimicrobial agent is applied to the nose contacting portion by spraying the effective amount of an antimicrobial agent onto the shield at the nose contacting portion.

In an embodiment of the present invention, the effective amount of an antimicrobial agent is applied to the nose contacting portion by spreading or wiping the effective amount of an antimicrobial agent onto the shield at the nose contacting portion.

In an embodiment of the present invention, the effective amount of an antimicrobial agent is applied to the nose contacting portion by depositing the effective amount of an antimicrobial agent onto the shield at the nose contacting portion.

In an embodiment of the present invention, the effective amount of an antimicrobial agent is applied to the shield nose contacting portion by depositing a powder form of the effective amount of an antimicrobial agent onto the shield at the nose contacting portion.

In an embodiment of the present invention, the effective amount of an antimicrobial agent is applied to the shield nose contacting portion by embossing the effective amount of an antimicrobial agent onto or into the shield at the nose contacting portion.

In an embodiment of the present invention, the effective amount of an antimicrobial agent is contained in fibers that are woven into the nose contacting portion of the shield.

In an embodiment of the present invention, the effective amount of an antimicrobial agent is contained in a layer of material that forms the nose contacting portion of the shield.

In an embodiment of the present invention, the shield further includes a nasal opening contacting portion adapted to directly contact nasal openings of the nose of the person, and said nasal opening contacting portion containing an effective amount of an antimicrobial agent capable of migrating from said nasal opening contacting portion into the person's nose, and effective to minimize or eradicate bacteria that colonize in the nose. The means of incorporating the antimicrobial agent are as discussed herein, and also include other methods known in the art for incorporating said agents, and related agents into substrates.

In an embodiment of the present invention, there is a clip for delivering an antimicrobial agent to the nasal mucosa lining the nostrils of a human nose. The clip comprises a pair of opposing arms connect to and extending outwardly from a common back portion. Each of the opposing arms are progressively inclined and/or biased towards each other in a coplanar fashion as each arm extends from the common back portion. Each of the opposing arms terminate at a distal end and form a gap existing between each of the distal ends. Each of the opposing arms has a nasal mucosa contacting surface. At least one of the common back portion, the opposing arms, or the nasal mucosa contacting surface are formed from, embedded with, impregnated with, coated with, and/or sheathed with a material containing an effective amount of antimicrobial agent capable of migrating to the nasal mucosa. The clip is applied to the nose such that the gap is occupied by the nasal septum separating each nostril and each of the arms contact the inside of the nostrils and the antimicrobial agent migrates from the clip to the nasal mucosa.

In some preferred embodiments of the present invention, the designated individual or group of individuals may be cultured to determine the effectiveness of the treatment, or otherwise determine whether the bacterial infection continues to exist.

In some preferred embodiments of the present invention, the application of the strip or mask may be in addition to other measures, procedures, or treatments directed at suppressing, mediating, eradicating, or reducing the spread of the bacterial infection among a population.

Additional features, advantages, and embodiments of the invention may be set forth or apparent from consideration of the following detailed description. Moreover, it is to be understood that both the foregoing summary of the invention and the following detailed description are exemplary and intended to provide further explanation without limiting the scope of the invention.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 is a schematic representation of a preferred embodiment of the method;

FIG. 2 is a schematic diagram of a preferred embodiment of the identification/exclusion/classification step of the method;

FIG. 3 is a schematic diagram of embodiments of the antimicrobial agent delivery mechanismused in the method;

FIG. 4 is a drawing of an embodiment mask of the present invention, an outside view;

FIG. 5 is a drawing of an embodiment mask of the present invention, an inside or rear view;

FIG. 6 is an inside or rear view of an embodiment mask of the present invention;

FIG. 7 is a front view of an embodiment clip of the present invention;

FIG. 8 is an isometric view of an embodiment clip of the present invention;

FIG. 9 is a partial schematic view showing an embodiment clip relative to a nose of a human.

FIG. 10 is a partial schematic view showing an embodiment clip attached to a nose of a human.

### DETAILED DESCRIPTION OF THE EMBODIMENTS

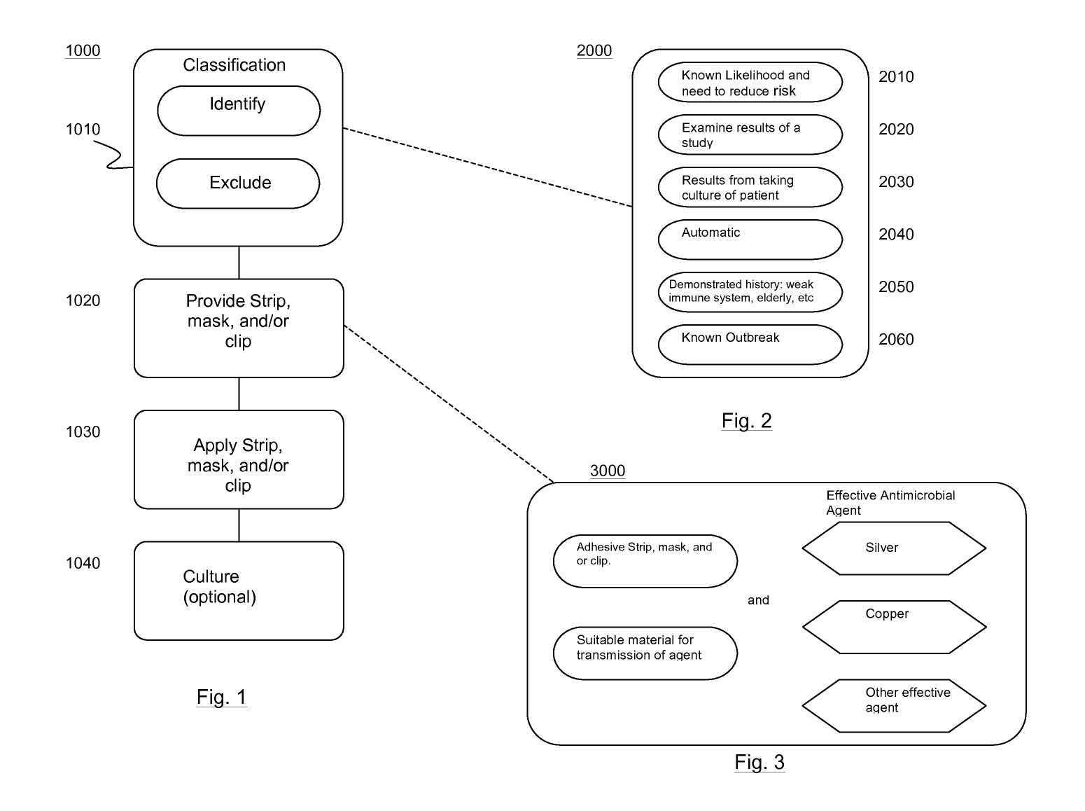

Referring to FIG. 1, the invention relates to a method for controlling the spread of bacterial infections within health care facilities, skilled nursing facilities and, institutional care facilities such as prisons, dormitories, military barracks, or anywhere people come in close proximity—increasing the incidence of the spread of infection among a population. Particularly, the invention concerns a method or protocol 1000 for reducing the spread of infection in the abovementioned situations by providing an adhesive strip, mask 11, and/or clip 31 containing silver or an other effective antimicrobial agent and applying the strip to the nose for dermal delivery, transdermal delivery, and/or other modes of transport to eradicate or inhibit the colonization of bacteria.

In a preferred embodiment of the present invention, the method includes step 1010 identifying a group of individuals who are carriers, or at risk of carrying or becoming carriers, of a bacterial infection that colonizes in the nose. Identification step 1010 may alternately include identifying an activity or location of an individual that puts said individual at risk of carrying the infection or becoming a carrier of said infection.

Referring to FIG. 2, exemplary identification sub-steps may include, identification from a known likelihood of incidence of a bacterial infection that colonizes in the nose, and a need to reduce the risk of contracting the infection 2010. Examples include nosocomial infections occurring at hospitals, skilled nursing facilities, or other health care institutions. Anywhere people come in close proximity generally increases the risk of spreading a bacterial infection. This includes dorms, military barracks, and camps. An identified activity, includes short hospital stays or outpatient services. According to the CDC, in 2008 a total of 182,000,000 patients entered US Hospitals as either a short-stay patient or for out-patient services, and were discharged from a hospital after a short-stay. Other targeted identification areas include areas of confined containment such as in planes, trains, and subways are identifiable risks for spreading the bacterial infection.

Similarly, identification may result from conducting a study, or examining the results of a study 2020 on incidences of bacterial infection propagation among a population. Examples include published or unpublished reports showing populations, activities, and/or locations that have a high incidence of infection resulting from colonization of the noses of carriers.

Direct identification is accomplished by taking cultures (step 2030) from patients or other individuals to determine whether they are infected or carriers of the targeted bacterial infection. Cultures may be ordered as a preoperative care to identify carriers. Healthcare workers and the family members of carriers may also be screened for MRSA or other bacterial infection. When an outbreak occurs, numerous screens may be performed to help identify the source of the infection. In some settings, such as nursing homes, a large number of people may be screened to evaluate the spread of colonization in a specific population.

In certain circumstances, identification of individuals, a group, or population for treatment with the strip, mask 11, and/or clip 31 is made automatically (step 2040), as part of a prophylactic approach to minimizing the likelihood of colonization. For example, newly admitted patients, new residents of nursing homes, care facilities, and patients receiving preoperative care, receive treatment with the strip without any previous cultures. Similarly, if the infection is found to be present among a population, automatic application to all patients (or members or classes of the population) may take place.

In other instances, identification is based on a demonstrated history of a population, group, or activity that results in a high incidence of infection and/or colonization (step 2050). For example, contact activities such a football or wrestling may have a demonstrated history of a high risk of transmitting MRSA, accordingly prophylactic treatment may be applied to these groups/activities.

In other instances, a known outbreak among a population or group, or individuals associated with a particular activity (step 2060) will lead to identification of candidates for treatment.

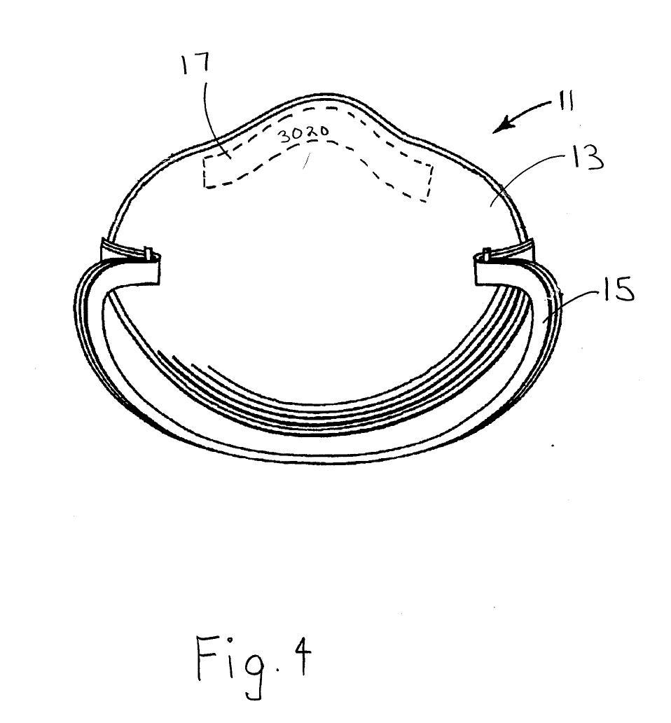

Referring to FIG. 3, there is shown a schematic relating to the antibiotic strip, mask 11, and/or clip 31, 3000 of the method 1000. Preferably, the strip is adhesive on at least one surface 3011 and is made of a material suitable for transmission 3013 of the antimicrobial agent 3020. Preferably, the agent 3020 comprises silver 3021. Alternately, the agent 3020 used on or in connection with the strip, mask 11, and/or clip 31 comprises copper 3022 or another transdermal antimicrobial agent 3023 capable of suppressing or eradicating the colonization of the targeted bacteria.

The mode of transport of the antimicrobial agent 3020 may occur dermally (over the skin surface), transdermally, by moisture and/or oils or fluids on or in the nose, and/or otherwise—so long as the mode achieves the desired result of delivering said antimicrobial agent 3020 from strip 19 and/or mask 11 to the colony of bacteria in, on and around the nose.

Silver—In a preferred embodiment, the antimicrobial agent 3020 used for delivery by the strip, mask 11, and/or clip 31, 3000 contains silver 3021. The strip, mask 11, and/or clip 31 may contain a plurality of layers having a silver coating 3021, or a compound containing silver 3021, which reacts with skin or moisture to release silver to the colony in the nose. The nose contacting portion 17 and/or the nasal opening contacting portion 21 may be constructed as discussed herein. Alternately at least one layer of the strip 3000 is impregnated with a silver containing compound 3021 capable of releasing silver ions when the strip is in contact with skin.

Alternate antimicrobial agents include copper 3022 or any other known antimicrobial agent 3023 capable of delivery from the strip, mask 11, and/or clip 31, 3000 to the bacteria.

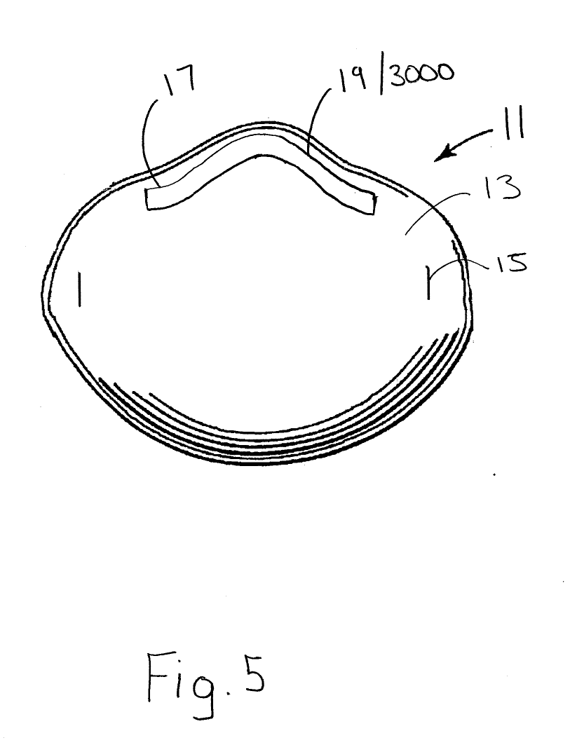

Referring to FIGS. 4 and 5, there is shown a mask 11 for covering the nose of a person. The mask 11 comprising an air filtering shield 13 constructed and configured to cover the nose and mouth of a person allowing the person to breathe filtered air through the shield 13. The shield typically includes a retainer 15 connected to the shield 13 and arrangeable about a person's head with the shield 13 retained against the person's face. Other known retainer means may be employed sufficient to retain the shield 13 against the person's face. The mask includes a nose contacting portion 17 that physically contacts a person's nose. The nose contacting portion 17 typically is that portion of the mask 11 that locates on the face above the lips and directly over the nose in contact with the nose and/or bridge of the nose. Said nose contacting portion 17 contains an effective amount of an antimicrobial agent 3020 (as discussed herein) capable of migrating from said nose contacting portion 17, to the colony of bacteria. As discussed herein, the antimicrobial agent 3020 is effective to eradicate or partially eradicate bacteria that colonize in the nose.

In an embodiment of the present invention, the nose contacting portion 17 of the shield 11 includes the adhesive strip 19/3000 applied to the shield 13. The adhesive strip 19/3000 containing an effective amount of an antimicrobial agent 3020 (as discussed in greater detail herein) capable of migrating from said nose contacting portion 17, to the colony of bacteria in and around the nose.

In an embodiment of the present invention, the adhesive strip 19/3000 is provided to a person to fix to the shield 13 of any commercially available mask. In other embodiments, the antimicrobial agent 3020 is applied prior to commercial distribution, or during the mask manufacturing process, as described herein.

In use, person places the mask 11 onto the person's face with the person's nose touching the contacting portion 17. The retainer 15 is then used to fix the mask 11 to the person's face to maintain contact of the person's nose with the nose contacting portion 17. In operation, the antimicrobial agent 3020 travels from the contacting portion 17 of the shield 13 to the colony of bacteria to eradicate, or partially eradicate a colony of bacteria located in the nose.

In an embodiment of the present invention, the effective amount of an antimicrobial agent 3020 is applied to the nose contacting portion 17 by spraying the effective amount of an antimicrobial agent 3020 onto the shield 13 at the nose contacting portion 17.

In an embodiment of the present invention, the effective amount of an antimicrobial agent 3020 is applied to the nose contacting portion 17 by spreading or wiping the effective amount of an antimicrobial agent 3020 onto the shield 13 at the nose contacting portion 17.

In an embodiment of the present invention, the effective amount of an antimicrobial agent 3020 is applied to the nose contacting portion 17 by depositing the effective amount of an antimicrobial agent 3020 onto the shield 13 at the nose contacting portion 17.

In an embodiment of the present invention, the effective amount of an antimicrobial agent 3020 is applied to the shield 13 nose contacting portion 17 by depositing a powder form of the effective amount of an antimicrobial agent 3020 onto the shield 13 at the nose contacting portion 17.

In an embodiment of the present invention, the effective amount of an antimicrobial agent 3020 is applied to the shield 13 nose contacting portion 17 by embossing the effective amount of an antimicrobial agent 3020 onto or into the shield 13 at the nose contacting portion 17.

In an embodiment of the present invention, the effective amount of an antimicrobial agent 3020 is contained in fibers that are woven into the nose contacting portion 17 of the shield 13.

In an embodiment of the present invention, the effective amount of an antimicrobial agent 3020 is contained in a layer of material that forms the nose contacting portion 17 of the shield 13.

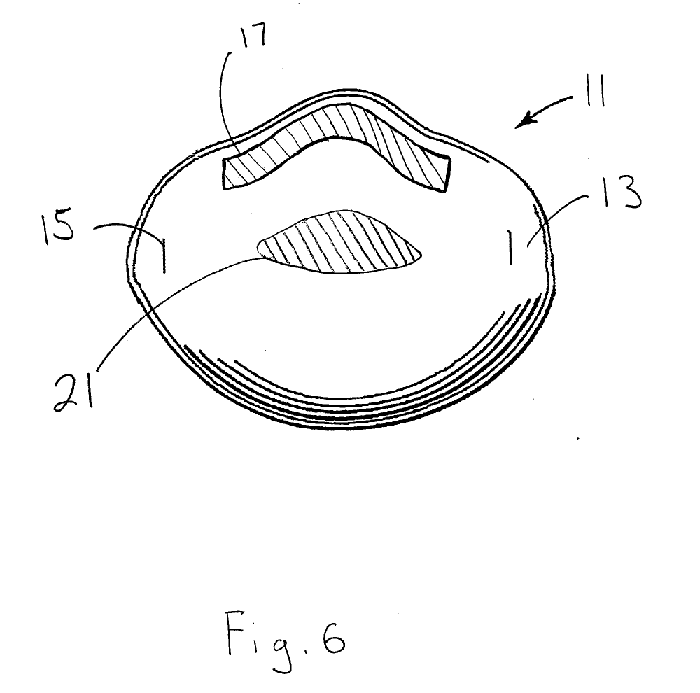

Referring to FIG. 6, the shield 13 further includes a nasal opening contacting portion 21 of the shield 13 adapted to directly contact nasal openings of the nose of the person. Said nasal opening contacting portion 21 containing an effective amount of an antimicrobial agent 3020 capable of migrating from said nasal opening contacting portion 21 into the person's nose, and effective to minimize or eradicate bacteria that colonize in the nose. The means of incorporating the antimicrobial agent 3020 are as discussed herein, and also include other methods known in the art for incorporating said agents, and related agents into substrates. In use, a person places the mask 11 onto the person's face with the person's nose located between the nose contacting portion 17 and the nasal opening contacting portion 17. The retainer 15 is then used to fix the mask 11 to the person's face. The person's nose is in contact with the nose contacting portion 17, and/or the person's nose structure forming the nasal openings of the person are in contact with the nasal opening contacting portion 17. In operation, the antimicrobial agent 3020 travels from the contacting portion 17 of the shield 13 through the nose, and/or the antimicrobial agent 3020 travels from the he nose contacting portion 17 to the colony of bacteria, to eradicate, or partially eradicate a colony of bacteria located in the nose.

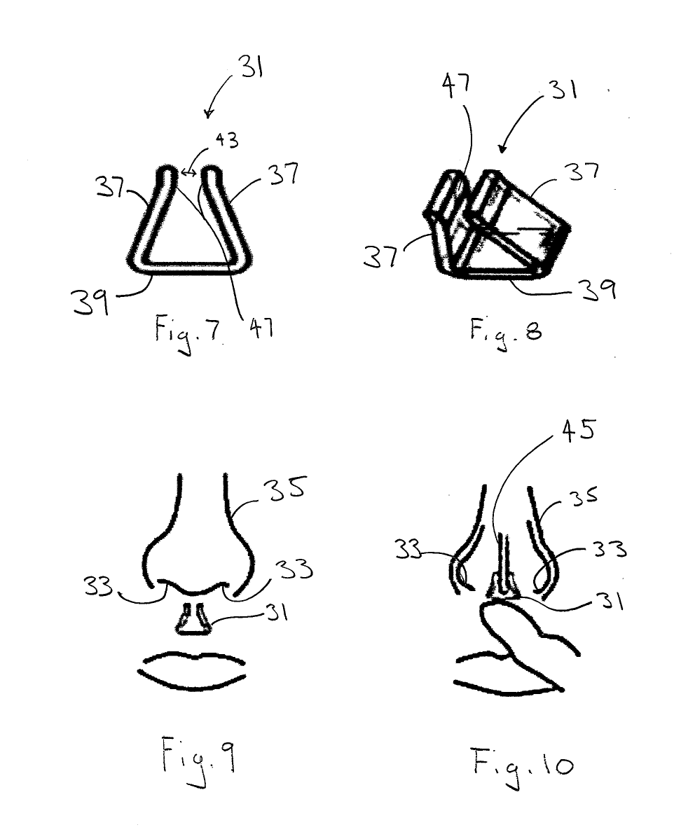

Referring to FIGS. 7-10, there is shown a clip 31 for delivering an antimicrobial agent to the nasal mucosa lining the nostrils 33 of a human nose 35. The preferred antimicrobial agent is as discussed herein, and is sufficient to migrate from the clip 31 to the inside lining/nasal mucosa of the nostrils 33.

The clip 31 comprises a pair of opposing arms 37 connected to and extending outwardly from a common back portion 39. Each of the opposing arms 37 are progressively inclined and/or biased towards each other in a coplanar fashion as each arm extends from the common back portion 39. Each of the opposing arms 37 terminate at a distal end 41 and form a gap 43 existing between each of the distal ends 41. Each of the opposing arms 37 has a nasal mucosa contacting surface 47. At least one of the common back portion 39, the opposing arms 37, or the nasal mucosa contacting surfaces 47 are formed from, embedded with, impregnated with, coated with, and/or sheathed with a material containing an effective amount of antimicrobial agent capable of migrating to the nasal mucosa. In a preferred embodiment, the antimicrobial agent is located at the nasal mucosa contacting surface 47.

In an embodiment of the present invention, at least one of the common back portion 39, the opposing arms 37, or the nasal mucosa contacting surfaces 47 are modified to increase delivery concentrations. Such modifications include texturizing the surface of the clip 31, embossing the surface of the clip 31, making small holes in the clip 31, or increasing the surface area of the clip 31 with or without increasing the cross sectional area of the clip 31 strictures discussed herein.

The clip 31 is applied to the nose 35 such that the gap 43 is occupied by the nasal septum 45 separating each nostril 33 and each of the arms 37 contact the inside of the nostrils 33 and the antimicrobial agent migrates from the clip to the nasal mucosa.

In an embodiment of the present invention clip 31, the arms 37 and/or the common back portion 39 are biased to resist widening of the gap. The biasing may be by nature of the flexibility/deflectability of the material used to construct the clip 31. The biasing may be accomplished by the shape of the clip 31. For example, the common back portion 39 can be arc or bight shaped. Any biasing sufficient to cause the arms 37 to straddle the nasal septum 45 and contact the lining of each of the nostrils 33 in a fixed manner that requires a force other than gravity to remove the arms of the clip from the nostrils 33. After the classification/identification of the population or group of individuals, application of the strip, mask 11, and/or clip 31 step 1030 is undertaken. Strips 19 or masks 11 are applied to the outside of the nose of those individuals identified in the classification. As discussed herein, in healthcare settings, the strip, mask 11, and/or clip 31 are applied to newly admitted patients, individuals who are admitted to elder care facilities, patients with weakened immune systems, patients receiving preoperative treatment, visitors, and possibly staff. In other settings, application of the strip, mask 11, and/or clip 31 is in reaction to an outbreak associated with a location or activity. In other settings, the strip, mask 11, and/or clip 31 is applied to individuals associated with an at risk activity such as being located in dense populations, or activities taking place in confined spaces, or where individuals are in close contact or proximity. Examples include traveling in trains, planes, subways, living in close quarters such as dorms, clinics, hospitals, military barracks, schools child care situations, and elder care situations. Other examples include sports activities where players are in close contact, such as basketball, football, and wrestling.

In some embodiments of the method, follow up screening of individuals is conducted by taking cultures of the nose, or other sites where infection is suspected. Follow up screening may also include other procedures known in the art at identifying symptoms associated with an active infection.

The invention may further include the step of providing MRSA education and training information to assist responsible individuals in identifying outbreaks and infection risks and undertaking a regimen, which may include the training and education on the application of the strip, mask 11, and/or clip 31, 1030. For example, in a university setting, university administrators are provided with education and training concerning the actual or perceived infection (MRSA, for example), how to identify, how to minimize an outbreak with proper sanitation procedures known in the art, how and when to use the strip, mask 11, and/or clip 31. Athletic directors and coaches are provided with said training on the infection, how to identify said infection, the risks of spreading the infection and methods of reducing the spread of infections. Similarly in child care and elder care situations, administrators and caregivers are provided with said education and training.

Although particular embodiments of the invention have been described in detail herein with reference to the accompanying drawings, it is to be understood that the invention is not limited to those particular embodiments, and that various changes and modifications, including the omission of steps or the inchangability of the order of steps, may be effected therein by one skilled in the art without departing from the scope or spirit of the invention.

What is claimed:

1. A clip for delivering an antimicrobial agent to the nasal mucosa lining the nostrils of a human nose, the clip comprising a pair of opposing arms connected to and extending outwardly from a common back portion, each of the opposing arms are progressively inclined and/or biased towards each other in a coplanar fashion as each arm extends from the common back portion, each of the opposing arms terminate at a distal end and form a gap existing between each of the distal ends, and each of the opposing arms has a nasal mucosa contacting surface, wherein at least one of the common back portion, the opposing arms, or the nasal mucosa contacting surface are formed from, embedded, impregnated, coated with, and/or sheathed with, a material containing an effective amount of an antimicrobial agent capable of migrating to the nasal mucosa, and wherein each of the arms are placed into the nostrils such that the gap is occupied by the nasal septum separating each nostril, each of the arms contact the nostrils and the antimicrobial agent migrates to said nasal mucosa.

2. The clip of claim 1, wherein the arms and/or the common back portion are biased to resist widening of the gap, thereby causing the arms to straddle the nasal septum and contact the lining of each of the nostrils in a fixed manner that requires a force other than gravity to remove the arms of the clp from the nostrils.

3. A method for controlling the spread of bacterial infections that colonize in the nose comprising:
identifying a group of individuals who are carriers, or at risk of carrying, or becoming carriers of a bacteria that colonizes in the nose,
providing a plurality of nasal clips having two arms that are biased towards each other of the two arms,
attaching the plurality of nasal clips, each to a person of the group of individuals identified, by inserting two arms of a nasal clip into the nostrils of the nose of each person such that the two arms straddle the nasal septum of the nose and each of the two arms contacts the lining of the nostrils, and
delivering an antimicrobial agent from the nasal clip to the lining of the nostrils.

4. A method of reducing the spread of bacterial infections at a location or facility and/or associated with an activity comprising the steps of:
classifying one or more individuals as targets for treatment,
providing a plurality of nasal clips capable of delivery of an antimicrobial agent from the nasal clip to nasal mucosa of the nostrils of the nose,
treating one or more individuals by the one or more individuals by inserting two arms of a nasal clip into the nostrils of the nose of each of the one or more individuals such that the two arms straddle the nasal septum of the nose and each of the two arms contacts the lining of the nostrils, and
eradicating or partially eradicating a colony of bacteria contained within or located near the nose of the one or more individuals treated.

5. The method of claim 4, further including the step of causing a release of an antimicrobial agent from one or more of the plurality of nasal clips to nasal mucosa of the nostrils of the nose to the nose of the one or more individuals.
